(19) United States
(12) Patent Application Publication
(10) Pub. No.: US 2005/0038509 A1
(43) Pub. Date: Feb. 17, 2005

(54) Valve prosthesis including a prosthetic leaflet

(76) Inventor: Kassem Ali Ashe, Mississauga, CA (US)

Correspondence Address: MOORE & VAN ALLEN PLLC, P.O. BOX 13706, Research Triangle Park, NC, 27709, US

(21) Appl. No.: 10/641,203

(22) Filed: Aug. 14, 2003

(51) Int. Cl. A61F 2/24

(52) U.S. Cl. 623/2.36; 623/2.38

(57) ABSTRACT

A prosthesis for repairing a damaged valve such as a heart mitral valve is disclosed. The prothesis comprises a prosthetic valve leaflet mounted on a frame. When implanted in a mitral valve, the leaflet replaces the function of an endogenous valve leaflet and coapts with an opposed endogenous valve leaflet. The prosthesis may further comprise a strut or other member to which the edge of an opposed valve leaflet can be sutured, and which can be used to correct deformities of a ventricle wall. Further disclosed is a method of repairing a damaged heart valve by providing a prosthetic valve leaflet or by providing a prosthetic valve leaflet and prosthetic attachment points for an opposed valve leaflet.

### FIELD OF THE INVENTION

The invention relates to valve prostheses, including but not limited to heart valve prostheses.

### BACKGROUND TO THE INVENTION

Human mitral valves comprise opposed anterior and posterior leaflets and a surrounding annulus of tissue. The movement of the free margins of the leaflets is limited by a series of chordae extending between the inner, ventricular face of the valve leaflets and the ventricle wall itself. During diastole, the leaflets fold back leaving the valve open and blood flows from the atrium, through the valve and into the ventricle. During systole, pressure within the ventricle forces the leaflets outward with outward flexure being limited by the chordae. Thus extended, the surfaces of the opposed leaflets coapt to prevent regurgitation of blood back into the atrium. The transition between open and closed valve positions is primarily due to movement of the larger, anterior mitral leaflet.

In many diseases, the mitral leaflets fail to properly coapt and there is resulting mitral regurgitation. There are a variety of causes for such failure, but generally it results from changes to the size of the annulus and damage to one of the opposed mitral leaflets or their supporting structures, the chordae, papillary muscles and ventricle wall.

Several surgical approaches currently exist to deal with mitral valve abnormalities: The valve may be replaced with a preserved human mitral valve, stented pig valves, or mechanical valves. U.S. Pat. No. 6,171,335 (Wheatley et al.) granted on Jan. 9, 2001, and U.S. Pat. No. 6,283,995 (Moe et al.) granted on Sep. 4, 2001, describe prosthetic valves which mimic a complete set of natural leaflets. However, preserved natural valves typically are expensive and have a short life. Conversely, mechanical valves typically require the administration of strong blood thinners.

A variety of known annuloplasty ring designs are used to correct deformities of the mitral valve annulus. These include rings known as Durand rings, Cosgrove-Edwards rings and Carpentier rings. Numerous designs are disclosed in the patent literature, for example, U.S. Pat. No. 5,061,277 (Carpentier et al.) granted on Oct. 29, 1991, U.S. Pat. No. 6,143,024 (Campbell et al.) granted on Nov. 7, 2000, and U.S. Pat. No. 6,187,040 (Wright) granted on Feb. 13, 2001.

Annuloplasty rings only remediate deformities of the annulus itself. Complex and technically demanding surgery typically is required to repair abnormalities of the leaflets and their supporting structures. Extensive deformity can make reconstruction of the leaflets impractical, and difficulty accessing the valve can make complex repairs unsafe. Replacement or repair of the supporting structures for the leaflets requires precise judgment and particularly skilled manipulation.

Accordingly, there is a need for a valve prosthesis suitable to restore near complete competence in the repair of all or most regurgitant lesions, which is readily available, durable and of low thrombogenicity. Such a device should preserve the sphincter action of the annulus, offer improved long term survival and not require a particularly high level of specialized skill for its successful use. The present invention is addressed to this need.

### SUMMARY OF THE INVENTION

In one aspect of the present invention, there is provided a valve prosthesis comprising a frame, the frame including a first portion extending longitudinally through an annular arc; and a substantially undeflectable leaflet extending downwardly from the first portion. The leaflet includes a prosthetic surface extending below and offset radially inwardly from the first portion. In a preferred embodiment, the surface is convex in cross-section planes transverse to the arc.

The prosthetic surface provides a surface for coapting with an opposed non-prosthetic leaflet. As will be seen herein, such a prosthesis may be adapted and used to repair a heart valve (and, accordingly, be referred to as a heart valve prosthesis). However, it may also be adapted and used to repair other types of valves.

The annular arc which is swept by the frame may be open-ended or closed. In one embodiment where the arc is closed, the frame includes a second portion which extends longitudinally as a chord of the annular arc.

There are situations where the prosthesis advantageously includes means extending downwardly from the frame for holding a suture at a position distanced away from both the frame and the prosthetic surface. In preferred embodiments for such situations, the prosthesis includes one or more struts extending away from the frame each to a distal end where a suture may be held.

The prosthesis has no moving parts and functions by facilitating the continued operation of a remaining endogenous valve leaflet, thereby improving effectiveness and durability. The incorporation of struts or other means for suture attachment in some embodiments facilitates correction of the attachment of the margin of the opposed endogenous leaflet to the ventricular heart wall, and can act as a prosthetic support for the wall.

The present invention also provides a heart valve prosthesis comprising a frame securable to a heart valve annulus, and a substantially undeflectable leaflet extending from said frame. The leaflet includes a prosthetic surface for coapting with an opposed non-prosthetic heart valve leaflet, and in desired situations may further include one or more struts or other means extending from the frame as described above for holding sutures. Such a prosthesis is considered particularly suitable for the repair of a heart mitral valve (where the non-prosthetic leaflet is then necessarily an anterior mitral valve leaflet).

In another aspect of the present invention, there is provided a method of repairing a mitral valve, the method comprising the step of attaching to the annulus of the mitral valve a leaflet having a prosthetic surface for coapting with an opposing mitral leaflet.

In a further aspect of the present invention, there is provided a method of repairing a mitral valve, the method comprising the step of replacing the posterior mitral leaflet with a leaflet having a prosthetic surface for coapting with an opposing anterior valve leaflet.

In certain situations, such methods advantageously may include the further step of limiting movement of the opposing leaflet with sutures.

The foregoing and other features and advantages of the present invention will now be described with reference to the drawings.

### BRIEF DESCRIPTION OF DRAWINGS

FIG. 1 is a front perspective view from above of a heart mitral valve prosthesis in accordance with the present invention.

FIG. 2 is a rear perspective view of the embodiment shown in FIG. 1

FIG. 3 is a top view of the embodiment shown in FIG. 1.

FIG. 4 is a sectional view taken along section line 4-4 in FIG. 3.

FIG. 5 is a front perspective view of another heart mitral valve prosthesis in accordance with the present invention.

FIG. 6 is a rear perspective view of the embodiment shown in FIG. 5.

FIG. 7 is a sectional view, partially cut-away, through both the prosthetic leaflet and one of the struts of the embodiment shown in FIG. 5.

FIG. 8 is a front perspective view of a further embodiment of the invention.

FIG. 9 is a rear perspective view of the embodiment shown in FIG. 8.

FIG. 10 is a diagrammatic view, partially cut-away, of the embodiment shown in FIG. 1 when implanted in a heart mitral valve. The valve is shown in a closed position.

FIG. 11 is a diagrammatic view as in FIG. 10, but with the valve now shown in an open position.

FIG. 12 is a diagrammatic view, partially cut-away, of the embodiment shown in FIG. 5 when implanted in a heart mitral valve. The valve is shown in a closed position.

### DETAILED DESCRIPTION

Three differing embodiments are illustrated in the drawings. Where like numbers are used to designate elements within the differing embodiments, it may be taken that the same or substantially the same element is present in the differing embodiments.

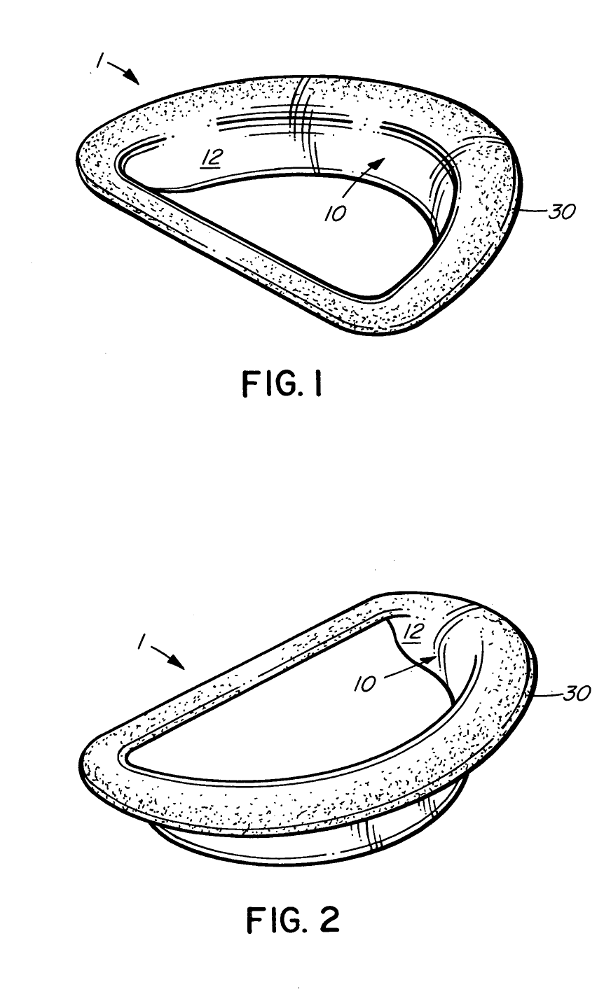

Embodiment Illustrated in FIGS. 1-4, 10-11

FIGS. 1-4 show a heart valve prosthesis generally designated 1. The prosthesis includes a leaflet generally designated 10 and a frame, the latter of which is comprised of a supporting ring 20 and padding 25 enclosed by a sheath 30. In FIGS. 1-3, ring 20 and padding 25 are hidden from view.

Ring 20 provides structural support. Padding 25 which is disposed around the periphery of ring 20 provides additional material through which sutures may be placed. Such padding is not considered essential, but its use is considered advantageous because it facilitates attachment of the prosthesis in a heart valve annulus with reduced risk of tearing surrounding tissue.

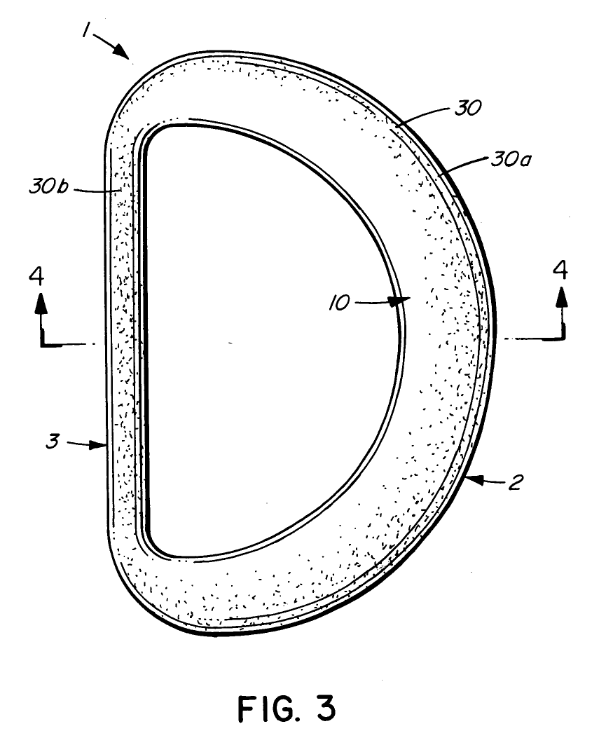

In FIG. 3, it will be seen that sheath 30 includes a first portion 30a that extends longitudinally through an annular arc and a relatively straight second portion 30b that extends longitudinally as a chord of the annular arc. Ring 20 and padding 25 (hidden from view in FIG. 3) are correspondingly shaped under the cover of sheath 30. Thus, it will be understood that frame (20, 25, 30) as a whole includes a first portion generally designated 2 that extends longitudinally through an annular arc and a relatively straight second portion generally designated 3 that extends longitudinally as a chord of the annular arc. Along portion 2, sheath 30 wraps partially around ring 20 and padding 25. Along portion 3, sheath 30 wraps fully around ring 20 and padding 25.

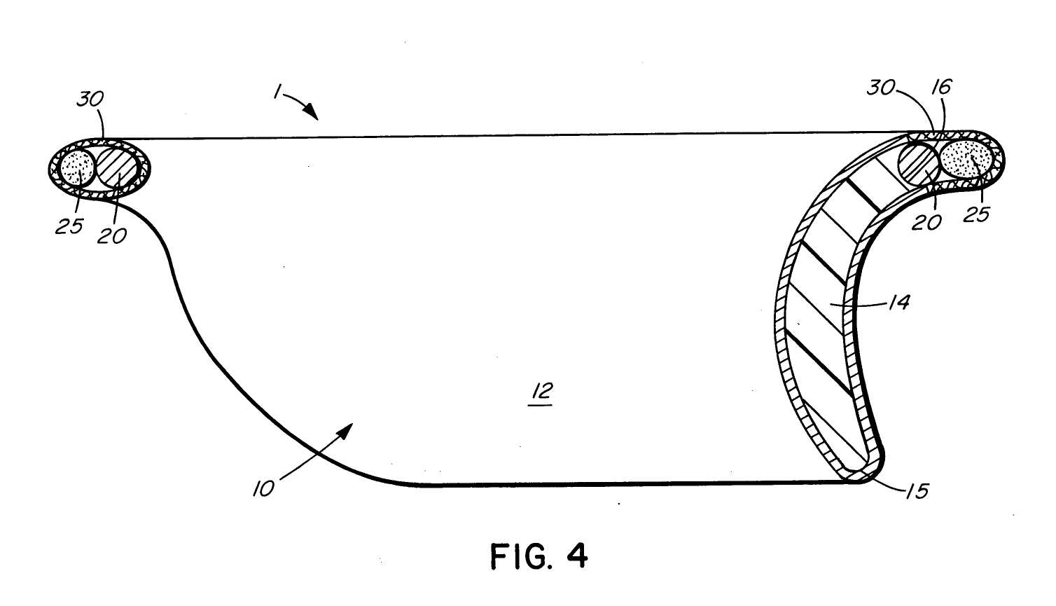

Leaflet 10 extends downwardly from the arcuate portion 2 of frame (20, 25, 30), and includes a prosthetic surface 12 which extends below and is offset radially inwardly from portion 2. As best seen in FIG. 4, surface 12 is convex in the plane of the cross-section which is depicted. That plane is transverse to the arc through which the frame extends. Although only the one transverse cross-section is shown, it will be understood that surface 12 is convex in a continuum of planes transverse to the arcuate portion of frame (20, 25, 30). Internally, leaflet 10 comprises a concavo-convex supporting skeleton 14 having a lower edge 15 and an upper edge 16, the latter of which is attached to ring 20. Externally, leaflet 10 is coated on opposed sides and around edge 16 with a biocompatible surface material 18.

Prosthesis 1 is sized for implant into the mitral valve annulus (not shown in FIGS. 1-4), leaflet 10 being sized such that surface 12 is operatively able to coapt with an opposed valve leaflet (likewise not shown in FIGS. 1-4). When leaflet 10 is used to replace an endogenous posterior leaflet, then approximately 50% of surface 12 typically should serve as operative coapting surface. It may also be noted then when appropriately sized, the outer perimeter of frame (20, 25, 30) typically will enclose an area roughly equal to that of the endogenous anterior leaflet of the valve being repaired.

While the general conformation of surface 12 as shown in FIGS. 1-4 presently is preferred, the fundamental requirements are that its shape and position be suitable for coaption with an opposed endogenous leaflet when in use, and to provide this function without adversely affecting the function of a mitral valve. Accordingly, it is contemplated that other conformations are possible. For example, in a cross-section corresponding to that shown in FIG. 4, a prosthetic surface conformed differently from that of surface 12 may have one or more areas of relative flatness rather than being smoothly convex.

To facilitate the implant procedure which is discussed below, frame (20, 25, 30) may have a limited degree of flexibility but, when implanted, it obviously must be sufficiently rigid to reliably hold leaflet 10 in a position where coaption will occur. Leaflet 10 is substantially undeflectable. That is to say that leaflet 10 is sufficiently rigid that when in use its position relative to frame (20, 25, 30) remains substantially constant.

Various suitable materials may be used in the manufacture of prosthesis 1. Preferably, ring 20 is made of a suitable metal such as titanium. Sheath 30 is made of a suitable flexible biocompatible material such as silicone rubber, polyester knit, ePTFE knit or PTFE knit. Skeleton 14 of leaflet 10 may be made of a suitable plastic or metal such as titanium, and may comprise a rigid skeleton framework rather than the solid body depicted in FIG. 4. A suitable coating material 18 is treated bovine pericardium or its equivalent. This may be seeded with cultured cells to present a more biologically compatible surface, but a range of alternatives will be apparent to those skilled in the art.

As is known to those skilled in the art, an important characteristic of any exposed surface used in heart valve prothesis is that it be biocompatible and present minimal risk of damage to blood cells. Bare plastic and metal surfaces generally are not considered to be suitable, and it is therefore desirable that they be suitably coated or sheathed.

The required dimensions of prosthesis 1 will depend upon the particular application, and will vary with the size and shape of the heart annulus in which it is to be implanted. Preferably, it is made available in a number of predetermined sizes and a user can select a suitable size from the range available. This approach is used with known annuloplasty prostheses.

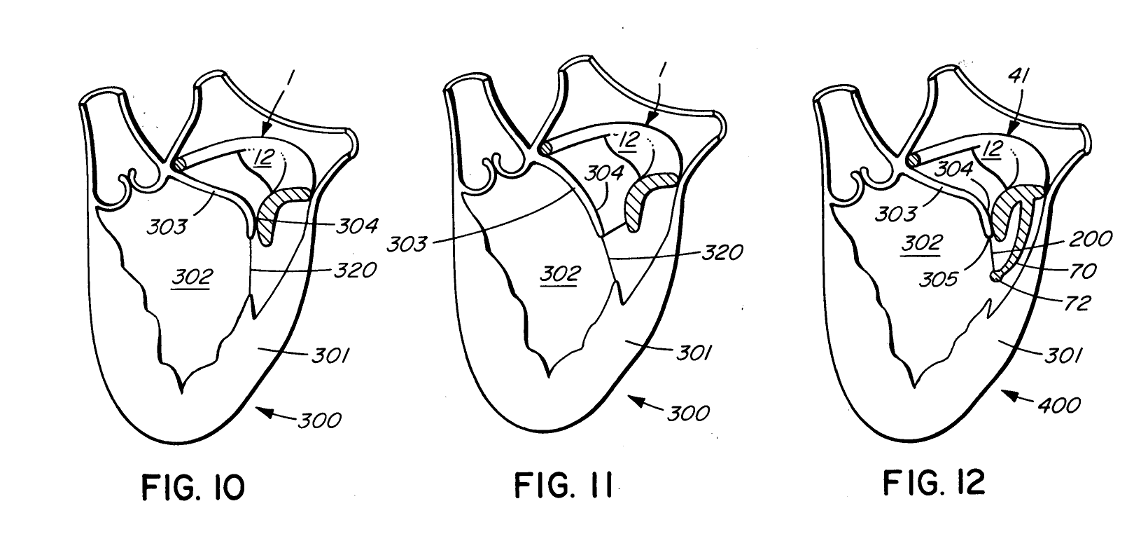

The use of prosthesis 1 will now be described with reference to FIGS. 10 and 11 which show the prosthesis implanted in the mitral valve of a heart generally designated 300. FIG. 10 depicts the repaired valve in a closed position during systole. FIG. 11 depicts the repaired valve in an open position during diastole.

Prosthesis 1 is secured within the annulus of the mitral valve by suturing. Sutures (not shown) are stitched through the material of the sheath 30 and padding 25 and through the supporting annulus tissue of heart 300 using routine methods well known to those skilled in the art. Heart 300 includes a ventricle wall 301 formed around a ventricular lumen 302 and an anterior leaflet 303 having an atrial surface 304. Movement of leaflet 303 is limited by chordae 320.

When ventricle wall 301 contracts during systole, pressure on blood within ventricular lumen 302 forces anterior leaflet 303 outwardly. As shown in FIG. 10, atrial surface 304 of anterior leaflet 303 then presses against and coapts with coapting surface 12 of leaflet 10. As ventricle wall 301 relaxes during diastole, blood flows inwardly through themitral valve. As indicated in FIG. 11, anterior leaflet 303, with its movement again restricted by chordae 320, then folds inward toward ventricular lumen 302 allowing blood to flow into the lumen past leaflet 10.

When prosthesis 1 is implanted into heart 300, the endogenous posterior leaflet (not shown) may be left in place and compressed down by suturing to the surrounding tissue, may be excised completely, may be left intact or may be used to supply suitable tissue for any necessary repairs to the opposing anterior leaflet. In general it is expected than any of these alternatives will be equally effective, but details of the installation may be adapted depending on the preference of the user and to suit specific circumstances. A particular advantage of the invention disclosed is that the endogenous posterior leaflet may be used as a ready source of tissue for repairing defects in the anterior mitral leaflet.

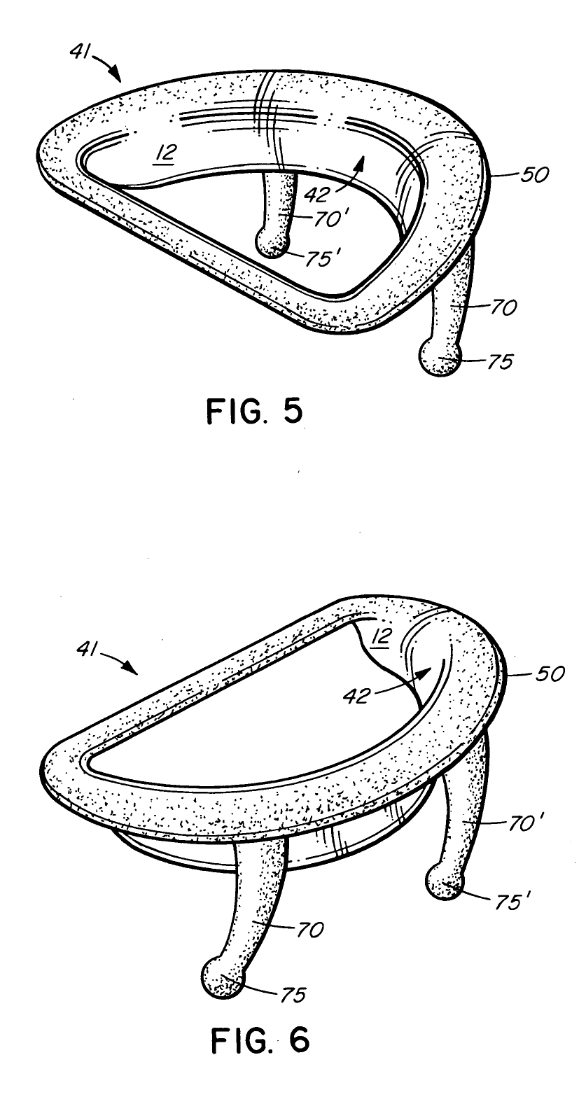

Embodiment Illustrated in FIGS. 5-7, 12

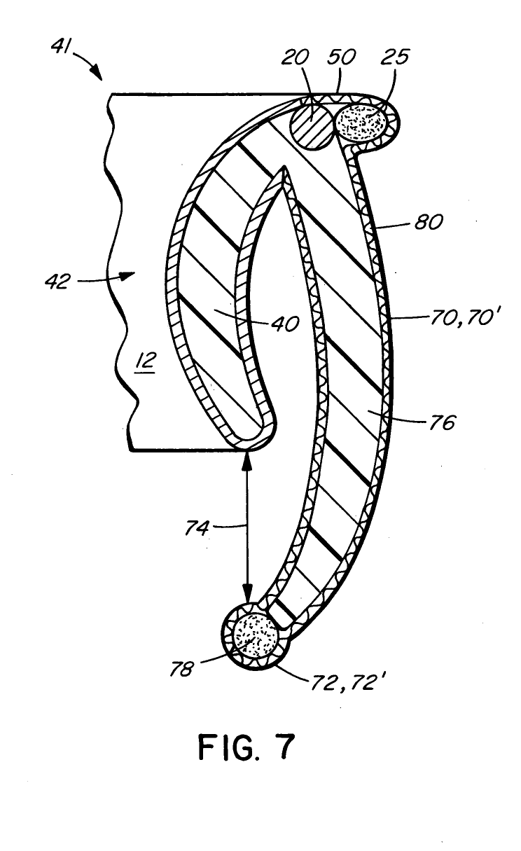

FIGS. 5-7 show a heart valve prosthesis generally designated 41. Except for minor modifications consequential to the addition of two projecting struts 70, 70′, and the struts themselves, the construction of prosthesis 41 is substantially the same as that of prosthesis 1. The structure includes a leaflet generally designated 42 with a prosthetic surface 12, a supporting ring 20 and padding 25. Ring 20 and padding 25 are enclosed by a sheath 50 and together comprise a frame (20, 25, 50). Leaflet 42 is substantially the same as leaflet 10 of prosthesis 1 except where it joins with struts 70, 70′ (see FIG. 7). Likewise, sheath 50 is substantially the same as sheath 30 of prosthesis 1 except where it forms around struts 70, 70′ (see FIG. 7).

Struts 70, 70′ are mounted on frame (20, 25, 50) and extend downwardly therefrom to distal ends 72, 72′ which are distanced away from both the frame and prosthetic surface 12. Preferably, ends 72, 72′ are positioned directly below at a distance 74 from surface 12. Also distance 74 should approximate the length of a normal heart chordae (not shown in FIGS. 5-7). In the case of humans, this distance typically may fall in the range of 13 mm to 30 mm.

As will be seen with particular reference to FIG. 7, struts 70, 70′ each include a supporting skeleton 76 formed integrally with supporting skeleton 40 of leaflet 42, and a generally spherical suture pad 78 at the lower end of skeleton 76. Although the integral formation is not essential to the function of the prosthesis, it assists to firmly fix the distance 74 between the lower edge of prosthetic surface 12 and the distal end 72, 72′ of each strut. Each strut, including its skeleton 76 and pad 78, is covered with a biocompatible material 80 preferably of the same type used for sheath 50.

Struts 70, 70′ each comprise a means extending downwardly from frame (20,25,50) for holding a suture (not shown in FIGS. 5-7) at a position distanced away from both the frame and prosthetic surface 12. The particular position in the embodiment shown is at the distal ends of the struts. However, it will be readily apparent to those skilled in the art that alternative means are possible and may be used to perform the same function. For example a strut may be structured to provide suture attachment positions at a number of alternative positions along it length, or along a continuum of possible positions, thereby allowing a user to select a preferred position for a given situation.

The use of prosthesis 41 will now be described with reference to FIG. 12 which shows the prosthesis implanted in the mitral valve of a heart generally designated 400. The repaired valve is depicted in a closed position during systole. For the purpose of the following discussion, heart 400 may be considered identical to heart 300 but for the absence of chordae 320 (see FIGS. 10-11) to limit the movement of anterior leaflet 303.

Just as prosthesis 1 is secured within the annulus of the mitral valve of heart 300, prosthesis 41 is secured within the annulus of the mitral valve of heart 400 by suturing. The essential added difference is that in heart 400 edge 305 of anterior leaflet 303 is attached to end 72 of strut 70 by means of sutures 200. The length of sutures 200 approximates that of distance 74 seen in FIG. 7. Although not shown, edge 305 at another point is likewise attached to end 72′ of strut 70′ by means of sutures.

During systole, the upper atrial surface 304 of anterior mitral leaflet 303 coapts with prosthetic surface 12 of the prosthetic leaflet 10. During diastole which is not illustrated, anterior leaflet 303 is drawn back by the flow of blood from atrium to ventricle in the same manner seen in FIG. 11.

By suturing edge 305 to ends 72, 72′, of struts 70, 70′, damaged chordae and support structures can be conveniently replaced. When sutures 200 are of a suitable length, the extent of outward flexure of the edge 305 of anterior leaflet 303 is limited such that when sutures 200 are fully extended as pressure is applied to blood on the ventricular side of prosthesis 41, then at least a portion of the upper atrial surface 304 of leaflet 303 proximate to edge 305 can coapt with the prosthetic surface 12.

A particular advantage of prosthesis 41 is the ease with which a suitable length for sutures 200 can be determined. In order to properly limit the outward movement of edge 305 of leaflet 303, the suture length should closely approximate distance 74. If such a length is used, then during systole edge 305 of anterior leaflet 303 necessarily will be approximately adjacent the lower edge of prosthetic surface 12 to allow proper coaptation between surface 12 and the upper atrial surface 304 of anterior leaflet 303.

The certainty of distance 74 is a significant advantage over known procedures, in which the appropriate length of sutures 200 must be estimated to take account of variation in the internal dimensions of ventricle wall 301 and papillae. Even if the morphology of the leaflets and their support structures are substantially normal, it requires a particularly high degree of skill and experience to judge an appropriate length for the sutures. In addition it can be extremely difficult to readily access the mitral valve well enough to visualize the structure and to determine an appropriate suture length and anchor points on the ventricle wall. The certainty of suture length in the use of prosthesis 41 can make an operating procedure relatively routine, reduce the skill necessary to successfully complete the operation, and reduce the time required to complete an operation. Risk to the patient is correspondingly reduced.

A further advantage of prosthesis 41 is that struts 70,70′ may be attached by suturing or like methods to the interior surface of the wall of the ventricle. In this manner, a strut may act as a prosthetic support for the ventricle wall and compensate for any dilation of the wall. Further, the strut may compensate for variations in the distance between the ventricle wall and the valve leaflets. Accordingly, the embodiment shown in FIGS. 5-7 and 12 is considered to be particularly suited to the repair of abnormalities which affect the support structures of the anterior mitral leaflet.

A number of variants to the structure shown in FIGS. 5-7 are possible. For instance, while the particular embodiment includes two struts, cases may arise where only one strut is desired or, alternately, where more than two struts are desired, and the position of such struts may differ from the positions shown in FIGS. 5-7. Further, it will be apparent to those skilled in the art that structures other than struts may be used to hold sutures in desired positions.

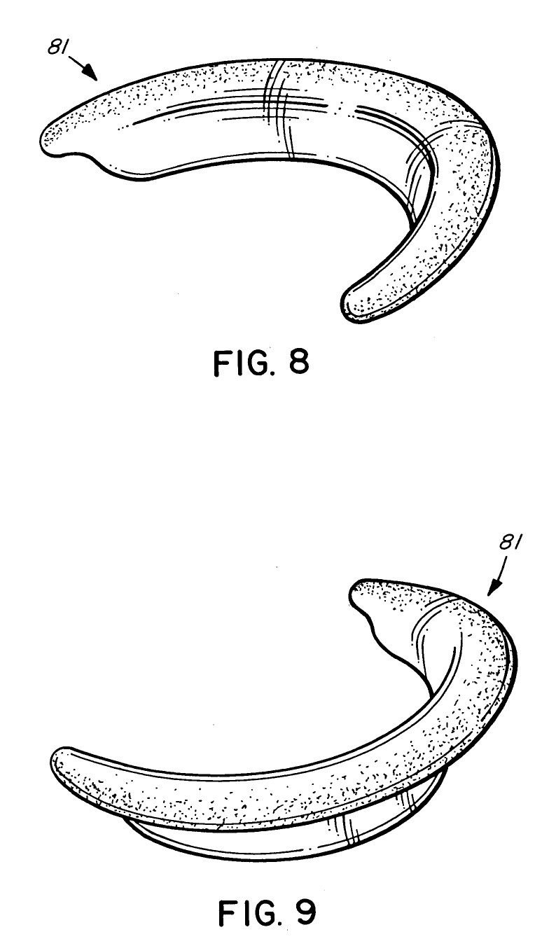

Embodiment Illustrated in FIGS. 8-9

FIGS. 8-9 show a heart valve prosthesis generally designated 81. The substantive difference between prosthesis 1 and prosthesis 81 is that the latter does not include a straight portion 3 (see FIG. 3) extending as a chord across the arced portion of the frame. A decision that a prosthesis does not require a straight portion 3 will depend upon the circumstances and will be a matter of judgment. In situations where it is determined that the portion of a valve annulus where straight portion 3 of prosthesis 1 normally would be sutured is not in need of correction, then prosthesis 81 may be used instead of prosthesis 1.

Although not illustrated in the drawings, it may be noted that the prosthesis shown in FIGS. 8-9 may be modified with struts or other means for holding a suture in the manner shown in FIGS. 5-7.

Although described here with reference to the human heart, it will be apparent to those skilled in the art that a prosthesis in accordance with the present invention may be adapted to repair other types of valves by making suitable adjustments to the shape and size of the device and to the disposition of its components so that it is able to coapt with an opposing valve leaflet.

Various modifications and changes to the embodiments that have been described can be made without departing from the scope of the present invention, and will undoubtedly occur to those skilled in the art. The invention is not to be construed as limited to the particular embodiments and should be understood as encompassing all those embodiments that are within the spirit and scope of the claims that follow.

1. A valve prosthesis, comprising:
(a) a frame, said frame comprising a first portion extending longitudinally through an annular arc; and,
(b) a substantially undeflectable leaflet extending downwardly from said first portion, said leaflet including a prosthetic surface extending below and offset radially inwardly from said first portion.

2. A valve prosthesis as defined in claim 1, said frame further comprising a second portion extending longitudinally as a chord of said annular arc.

3. A valve prosthesis as defined in claim 1, wherein said surface is convex in cross-section planes transverse to said arc.

4. A valve prosthesis as defined in claim 3, said frame further comprising a second portion extending longitudinally as a chord of said annular arc.

5. A valve prosthesis as defined in claim 1, 2, 3 or 4, wherein said prosthesis is a heart valve prosthesis.

6. A valve prosthesis as defined in claim 1, 2, 3 or 4, further including means extending downwardly from said frame for holding a suture at a position distanced away from both said frame and said prosthetic surface.

7. A valve prosthesis as defined in claim 1, 2, 3 or 4, further including means extending downwardly from said frame for holding a suture at a position distanced away from both said frame and said prosthetic surface, and wherein the minimum distance between said position and said prosthetic surface is in the range of 13 mm to 30 mm.

8. A valve prosthesis as defined in claim 1, 2, 3 or 4, further including a strut extending downwardly from said frame to a distal end distanced away from said frame and said prosthetic surface.

9. A valve prosthesis as defined in claim 1, 2, 3 or 4, further including a strut extending downwardly from said frame to a distal end distanced away from said frame and said prosthetic surface, and wherein the minimum distance between said distal end and said prosthetic surface is in the range of 13 mm to 30 mm.

10. A heart valve prosthesis, comprising:
(a) a frame securable to a heart valve annulus; and,
(b) a substantially undeflectable leaflet extending from said frame, said leaflet including a prosthetic surface for coapting with an opposed non-prosthetic heart valve leaflet.

11. A heart valve prosthesis as defined in claim 10, wherein said heart valve is a mitral valve and the non-prosthetic valve leaflet is an anterior mitral leaflet.

12. A heart valve prosthesis as defined in claim 10 or 11, further including means extending downwardly from said frame for holding a suture at a position distanced away from both said frame and said prosthetic surface.

13. A heart valve prosthesis as defined in claim 10 or 11, further including means extending downwardly from said frame for holding a suture at a position distanced away from both said frame and said prosthetic surface, and wherein the minimum distance between said position and said prosthetic surface is in the range of 13 mm to 30 mm.

14. A heart valve prosthesis as defined in claim 10 or 11, further including a strut extending downwardly from said frame to a distal end distanced away from said frame and said prosthetic surface.

15. A heart valve prosthesis as defined in claim 10 or 11, further including a strut extending downwardly from said frame to a distal end distanced away from said frame and said prosthetic surface, and wherein the minimum distance between said distal end and said prosthetic surface is in the range of 13 mm to 30 mm.

16. A method of repairing a mitral valve comprising attaching to the annulus thereof a prosthetic surface for coapting with an opposing mitral leaflet.

17. A method of repairing a mitral valve comprising replacing the posterior mitral leaflet with a prosthetic surface for coapting with an opposing anterior valve leaflet.

18. A method as described in claim 16 or 17, wherein movement of said opposing leaflet is limited by sutures.
